(19) United States
(12) Patent Application Publication
(10) Pub. No.: US 2023/0310682 A1
(43) Pub. Date: Oct. 5, 2023

(54) APPARATUS FOR DISINFECTING AT LEAST ONE ROOM, IN PARTICULAR A DWELLING ROOM FOR PERSONS, WITH AN ATOMISER

(71) Applicant: Dürr Systems AG, Bietigheim-Bissingen (DE)

(72) Inventors: Patrick Häussermann, Korb (DE); Bernhard Seiz, Lauffen (DE); Kevin Preuss, Mühlacker (DE)

(21) Appl. No.: 18/005,764

(22) PCT Filed: Jul. 16, 2021
(86) PCT No.: PCT/EP2021/069943

§ 371 Date: Jan. 17, 2023

(30) Foreign Application Priority Data
Jul. 27, 2020 (DE) 10 2020 119 714.4

(51) Int. Cl. A61L 2/22; A61L 2/26; B05B 3/10; B05B 3/00; B05B 7/00; B05B 15/65

(52) U.S. Cl. CPC A61L 2/22; A61L 2/26; B05B 3/1014; B05B 3/1035; A61L 2202/16; B05B 7/0093; B05B 15/65; A61L 2202/25; A61L 2202/15; B05B 3/001

(57) ABSTRACT

The invention relates to an apparatus (100) for disinfecting at least one room, in particular for one or more persons, preferably a dwelling room, storage room, cooling room or treatment room, for example a treatment room of a building, in particular a sickroom, a patient room and/or an operating room, with a rotatable bell cup (1) for atomising a disinfectant, a hollow drive shaft (10) for driving the bell cup (1), and a housing (11) in which a drive unit (2) for driving the drive shaft (10) is accommodated and preferably the drive shaft (10) is accommodated. The apparatus (100) is particularly characterised in that the housing (11) has a plurality of cooling ribs (12), and/or the drive shaft (10) is provided with a labyrinth seal (13), preferably in order to prevent unwanted penetration of liquid into the housing (11).

The invention relates to an apparatus for disinfecting at least one room, preferably for one or more persons, for example a dwelling room, storage room, cooling room and/or treatment room, for example a treatment room of a building, in particular a sickroom, a patient room and/or an operating room, with an atomiser expediently for atomising a disinfectant into the room.

WO 2019/238503 A1 discloses an apparatus for disinfecting an operating room, for example. The apparatus includes, in particular, a rotatable bell cup for atomising a disinfectant such as hypochlorous acid (HOCl) into the operating room and a hollow drive shaft for driving the bell cup, the disinfectant being supplied to the bell cup through the hollow drive shaft. Depending on the operation of the apparatus, high temperatures may possibly occur (for example, above 80° C.), which may negatively affect the temperature-sensitive hypochlorous acid. Hypochlorous acid is also highly corrosive. Therefore, a particular challenge is to protect sensitive components from the hypochlorous acid, but also from other, possibly less corrosive liquids such as rinsing agents, room humidity, etc.

A task of the invention is to provide an improved and/or alternative apparatus for disinfecting at least one room, preferably accessible by one or more persons.

This task can be solved with the features of the independent claim. Advantageous further developments of the invention can be found in the dependent claims and in the following description of preferred embodiments of the invention.

The invention relates to an apparatus for disinfecting at least one room, for example a room for one or more persons, preferably a dwelling room, storage room, cooling room and/or treatment room (expediently for humans or animals), for example a treatment room of a building, in particular a sickroom, a patient room and/or an operating room, for example in a hospital or doctor's surgery. However, the dwelling room may also include, for example, classrooms, bathrooms, toilets, sanitary facilities or compartments of aircrafts, ships or other vehicles. The dwelling room may preferably also include a veterinary dwelling room and/or a waiting room.

The apparatus comprises a rotatable bell cup (in particular a so-called high rotation bell) for atomising a disinfectant, in particular into the room, and a hollow drive shaft for driving the bell cup.

The apparatus includes a (for example, substantially cylindrical) housing in which, for example, a drive unit (for example, a motor) for driving the drive shaft can be at least sectionally accommodated. The housing is thus in particular a drive unit housing (for example, a motor housing). Alternatively or additionally, the drive shaft in particular can be accommodated at least sectionally in the housing.

The drive unit can in particular comprise a motor for driving the drive shaft and thus in particular the bell cup, for example, an electric motor, an electric turbine and/or a (for example, pneumatically driven) turbine, in particular an air turbine.

The apparatus is characterised in particular in that the housing has a plurality of cooling ribs (for example, heat ribs or cooling fins) and can thus preferably function as a heat exchanger. In particular, this allows heat to be effectively dissipated from the interior of the housing to the outside, for example so as to be able to maintain temperatures inside the housing at a level suitable for the disinfectant in particular.

The apparatus is alternatively or additionally characterised in particular in that the drive shaft is provided with a (in particular contactless) labyrinth seal, for example in order to prevent unwanted penetration of liquid such as disinfectant and/or rinsing agent (but also room humidity, for example) into the housing. The labyrinth seal can be disposed in particular at the exit of the drive shaft from the housing.

The apparatus is alternatively or additionally characterised in particular by a ring element made of a material for liquid intake, for example in order to prevent or at least delay unwanted penetration of liquid (such as disinfectant and/or rinsing agent) into the housing.

It is possible that the cooling ribs project, for example, radially laterally outwards from the housing, and/or are spaced substantially uniformly from each other in the circumferential direction of the housing.

The cooling ribs may, for example, sectionally extend laterally outside along the drive shaft and/or substantially parallel to the drive shaft.

It is possible for the cooling ribs sectionally to extend laterally outside along the drive unit, for example so as to be able to effectively dissipate the heat generated by the drive unit.

It is possible that the housing and/or the cooling ribs have a recess for receiving a part of the drive unit. This allows, for example, a compact housing with an (expediently at least partially) integrated drive unit.

The apparatus may, for example, have a blower (for example, a fan, in particular an axial fan) for generating a gas flow, preferably in order to transport and/or distribute disinfectant atomised by means of the bell cup in the room.

The blower is preferably located upstream of the housing and/or the bell cup.

It is possible that gas guiding channels are disposed between the cooling ribs and that the gas guiding channels serve to guide the gas flow. The cooling ribs may preferably form lateral guide elements of the gas guiding channels.

The gas guiding channels may preferably be used to guide the gas flow, for example towards the bell cup and/or the atomised disinfectant.

The blower and/or the gas flow can thus preferably be used to distribute atomised disinfectant in the room and/or to dissipate heat from the housing, in particular via the cooling ribs.

Since the drive shaft rotates during operation, a static seal and/or contact seal, for example, can prove negative.

The labyrinth seal may therefore preferably be configured as a contactless labyrinth seal.

The labyrinth seal may possibly comprise a first (preferably annular) section rotatable with the drive shaft, and/or a second (preferably annular) static section. The static section is characterised, for example, in that it does not rotate with the drive shaft during operation.

The first section is preferably associated with the drive shaft, being, for example, mounted (expediently directly or indirectly) on the drive shaft.

The second section is preferably associated with the housing, being, for example, mounted (expediently directly or indirectly) on the housing (for example on one or more cooling ribs).

It is possible that the labyrinth seal performs its sealing function not until and/or only when the drive shaft rotates and in particular not until and/or only when the first section rotates. When no rotation is taking place, on the other hand, the labyrinth seal preferably does not ensure a sealing function.

It is possible that an (expediently relatively thin) intermediate gap is disposed between the first section and the second section, so that the first section and the second section preferably do not touch.

The intermediate gap can, for example, extend into the interior of the housing, for example to a bearing for the, in particular, rotatable mounting of the drive shaft.

It is possible that (expediently during operation of the apparatus) an air barrier is formed in the intermediate gap due to rotation of the first section and preferably the air barrier acts as a seal.

It is thus possible that the labyrinth seal is inactive when the drive shaft is not rotating (and thus in particular when the first section is not rotating) and/or is only brought into a state activated by means of the air barrier when the drive shaft rotates (and thus in particular only when the first section is rotating).

The labyrinth seal can thus be configured, for example, so as to be active only when the drive shaft, and thus in particular the first section, is rotating.

Therefore, the apparatus can be configured, for example, to rotate the drive shaft even if no atomisation of disinfectant is taking place, in particular so as to prevent liquid (for example disinfectant and/or rinsing agent) present on the labyrinth seal from penetrating into the housing (for example to a bearing, etc.) via the labyrinth seal.

It is possible that the first section and the second section do not touch each other, expediently at least not for sealing purposes.

It is possible that at least one, for example, elastic (for example, spring, rubber etc.) loading element is disposed between the second section and a bearing (in particular accommodated in the housing) for in particular rotatable mounting of the drive shaft, in order to act on the bearing. The loading element serves in particular for pre-tension of the bearing and/or for tolerance compensation of the bearing.

The loading element can in particular be mounted on or in the second section.

A hollow, preferably non-co-rotating inner pipe for supplying disinfectant may possibly be disposed in the drive shaft. The inner pipe is preferably static, so that it does not rotate during operation, in particular, it does not co-rotate with the drive shaft.

The drive shaft and the inner pipe are preferably disposed coaxially with one another.

The inner pipe can comprise, for example, a nozzle through which disinfectant can be discharged.

The disinfectant can, for example, be supplied (in particular indirectly) to the bell cup via a rotatable plate, in particular a deflector plate or distributor plate.

The plate can be expediently accommodated in the bell cup, for example.

The inner pipe may, for example, have a first portion and a second portion.

Due to the rotation of the drive shaft, the disinfectant-filled inner pipe, which does not rotate during operation, can also be stressed by vibrations. The design of the inner pipe with the first portion and the second portion may therefore be adapted in particular to system vibrations (for example, a natural frequency of the system) occurring during operation.

The second portion may, for example, have a greater external diameter and/or a greater wall thickness than the first portion. Alternatively or additionally, the second portion may have a greater passage cross-section for the disinfectant than the first portion.

The first portion may, for example, have an outlet opening for the disinfectant. The outlet opening may, for example, be part of a nozzle mounted and/or disposed on the first portion.

The nozzle preferably serves for pressure increasing and can have an outlet diameter of, in particular, 0.5 mm<=d<=0.9 mm.

The second portion may, for example, run between the drive unit and/or be carried in a mount for the housing.

Between the first portion and the second portion, for example, at least one stepped offset may be disposed.

It is possible that the labyrinth seal is disposed on the head-sided front side, in particular on the end of the housing facing the bell cup. The labyrinth seal can thus be configured in particular to prevent liquid (for example, disinfectant and/or rinsing agent) from penetrating the housing from above.

It is possible that the housing is (expediently directly or indirectly) carried on a mount and the mount preferably forms a floor for the housing.

An O-ring seal may, for example, be disposed between the mount and the housing, in particular to seal the housing, for example on the floor side. Alternatively or additionally, a static part of the drive unit can be provided with an O-ring seal, in particular to seal the housing, for example on the floor side. It is possible that, for example, an O-ring seal is placed between the mount and the inner pipe.

It is possible that an inner face of a static part of the drive unit is protected by an inner tubular element made of corrosion-resistant plastic or by a corrosion-resistant coating.

The static part of the drive unit preferably represents a stator of the drive unit.

The apparatus can, for example, have an (expediently annular) brush seal, preferably an anti-static brush seal and/or carbon fibre brush seal, for example in order to seal the housing, in particular on the floor side.

The brush seal is preferably attached to the mount, in particular statically.

It is possible that, in particular, a rotatable, part of the drive unit is provided with the brush seal.

It is possible that the apparatus includes a ring structure.

The ring structure may, for example, be disposed between the housing and the bell cup.

The ring structure can serve, for example, to reduce noise, in particular as a noise reduction skirt. Noise reduction can be achieved in particular by reducing turbulences on the outer face of the bell cup.

The ring structure can be used, for example, to encase and/or cover a base of the bell cup, a part of an outer face of the bell cup and/or the labyrinth seal.

The ring structure can, for example, serve as a gas conducting element in order to conduct further a gas flow generated by means of the blower, for example towards the bell cup and/or towards the atomised disinfectant.

The ring structure can, for example, be openable and/or be composed of a plurality of, in particular at least two, sections (for example half shells), in particular so as to be able to remove the ring structure from the apparatus and, if necessary, re-mount it on the apparatus.

It is possible that the ring structure comprises at least two sections releasably mountable to with each other.

The ring structure can, for example, be openable and/or closable by means of at least one expediently releasable latch, plug and/or clamp connection. The latch, plug and/or clamp connection can, for example, be configured as a snap and/or hook connection or, for example, be combined therewith.

The ring structure can, for example, be configured as a hollow-chambered or solid profile.

The ring structure can, for example, have a concave and/or laterally outwards extending outer face, preferably in order to conduct further the gas flow generated by the blower laterally outwards, in particular towards the bell cup and/or towards the atomised disinfectant.

It is possible that the outer face is directed towards a spray edge of the bell cup with a deviation of less than +/−30°, 25°, 20° or 15°.

It is possible that the ring structure has a, for example, integrated rinsing agent channel, for example for connecting to a rinsing agent line and/or for supplying rinsing agent.

The rinsing agent channel can, for example, have an inlet hole and at least one outlet hole.

It is possible that the inlet hole is disposed in the outer face and preferably has a connecting device a connecting device for connecting to the rinsing agent line. Alternatively or additionally, the at least one outlet hole can be disposed, for example, in an inner face of the ring structure and/or be directed towards the bell cup.

The outer face preferably forms an (in particular radially) outer lateral surface of the ring structure.

The inner face preferably forms an (in particular radially) inner lateral surface of the ring structure.

The ring structure can, for example, have a side wall construction which can preferably run annularly.

The rinsing agent channel can extend, for example, in the side wall construction and/or extend, for example, through the side wall construction.

It is possible that the bell cup, the drive shaft and/or the labyrinth seal on the one hand and the ring structure on the other hand delimit a (for example annular-gap-shaped) intermediate space.

The intermediate space can, for example, have a (preferably annular) inlet opening so that, for example, liquid (for example, disinfectant, rinsing agent and/or room humidity etc.) can penetrate into the intermediate space. The inlet opening can, for example, be delimited by the bell cup on the one hand and the ring structure on the other hand and/or face the bell cup.

It is possible that the rinsing agent channel, for example, with its at least one outlet hole, opens out into a preferably annular-gap-shaped intermediate space.

The intermediate space can be formed, for example, between the ring structure on the one hand and the bell cup, the drive shaft and/or the labyrinth seal on the other hand.

It is possible that at least one exit opening is formed, through which liquid (for example, rinsing agent and/or disinfectant) can drain out of the intermediate space, preferably by gravity, whereby flooding of the intermediate space can preferably be prevented.

It is possible that the at least one exit opening is formed in the ring structure, preferably in a lower base portion and/or base portion of the ring structure facing away from the bell cup. However, embodiments are possible in which the at least one exit opening is not formed in the ring structure.

It is possible that the housing with the cooling ribs is surrounded by a (preferably substantially cylindrical) enclosure.

The enclosure can extent, for example, laterally spaced from the cooling ribs.

The gas flow can be guided through the enclosure, for example, so that it can be expediently guided to the bell cup and/or to the atomised disinfectant.

The bell cup and/or the ring structure can preferably be disposed outside the enclosure.

It is possible that the drive shaft, the inner pipe, the housing and/or the enclosure are disposed substantially coaxially and/or are substantially cylindrical in shape.

It is possible that at least one leakage outlet (for example on the floor side) is disposed in the housing and/or in the mount, preferably so as to be able to drain off highly corrosive liquid from the housing.

A leakage discharge line, for example, can be attached to the at least one leakage outlet.

If, for example, liquid penetrates into the area of the drive unit, a leakage occurs at the inner pipe and/or there is a backflow of liquid from the nozzle, the in particular highly corrosive liquid can be drained off through the at least one leakage outlet.

It is possible that the drive shaft and at least one of the following parts are configured as a jointly balanced unit: the bell cup, a deflector plate or distributor plate of the bell cup, the rotatable part of the drive unit and/or the first section of the labyrinth seal.

As previously mentioned, it is possible for the apparatus to have a mount for the housing on which the housing can be expediently placed on and/or mounted.

For example, at least one supply line for liquid (for example, disinfectant and/or rinsing agent) can run in the mount, through which the liquid can be fed into the inner pipe, for example. This allows a medium, in particular a liquid, to be expediently fed into the inner pipe from the outside.

For example, at least one control line for controlling the drive unit can run in the mount. Alternatively or additionally, at least one drive line for the drive unit can run in the mount, for example a current line, a compressed air line, etc.

As previously mentioned, the labyrinth seal is preferably inactive during non-rotating drive shaft and thus non-rotating first section.

The apparatus can therefore have, for example, a control device which is configured to allow the drive shaft, and thus in particular the first section of the labyrinth seal, to post-rotate for a predefined period of time after an atomisation process and/or after a rinsing process of the intermediate space, whereby the labyrinth seal (in particular its air barrier) can preferably be kept in an activated state, for example until any liquid which may be present in the intermediate space has been able to drain off, in particular via the at least one exit opening.

The control device can also be configured to allow the drive shaft and thus in particular the first section of the labyrinth seal, for example during a rinsing process for rinsing the intermediate space, to rotate, whereby the labyrinth seal (in particular its air barrier) can preferably be kept in an activated state.

The disinfectant is preferably a hypochlorous acid (HOCl).

The hollow drive shaft, the bell cup, the labyrinth seal, the housing, the inner pipe, the ring structure and/or the mount can be made of metal, in particular of one or more metals which are resistant to highly corrosive media such as hypochlorous acid.

The ring structure, for example, can also be made of a suitable plastic.

The housing may optionally have, for example, a plurality of cooling ribs (for example, heat ribs or cooling fins). Alternatively or additionally, the drive shaft can optionally be provided with, for example, a labyrinth seal.

It is possible that the apparatus comprises a ring element (in particular a liquid-intake ring element, for example a sealing element) made of a material for liquid intake, for example in order to advantageously prevent or at least delay unwanted penetration of liquid into the housing.

The material preferably has a capacity (in particular a material capacity) and/or a material property for (in particular high) liquid intake.

The material may comprise, for example, a fibrous structure, in particular a non-woven fibrous structure, a foamed plastic and/or a felt.

The material can, for example, be designed as a liquid-absorbing material (in particular as a disinfectant-absorbing and/or rinsing agent-absorbing material).

The material is preferably designed to take in, to store (expediently to hold) and/or to allow liquid to dry by expediently natural evaporation.

It is possible that an external diameter of the ring element is smaller than an internal diameter of the drive shaft and/or that the ring element forms a static component, so that preferably the ring element, like the inner pipe, does not undergo any rotational movement.

For example, the apparatus may have a nozzle, preferably with an outlet opening (for example a nozzle outlet) for discharging disinfectant.

The ring element and/or the nozzle is preferably demountable and replaceable.

It is possible that the bell cup has a rotatable plate in its centre, in particular a deflector plate and/or distributor plate.

It is possible that the plate is releasable and expediently demountable, preferably by means of an expediently suitable tool, so that the (in particular underlying) nozzle and/or the (in particular underlying) ring element can be advantageously reached and preferably replaced.

The nozzle, in particular its outlet opening, is preferably configured to discharge (for example, to spray) disinfectant from preferably the inner pipe and/or onto the rotatable plate.

The nozzle and/or the outlet opening can in particular be configured to discharge (for example, to spray) the disinfectant onto in particular an underside of the plate, from where the disinfectant can be transported, for example, onto the inner face of the bell cup.

The nozzle may, for example, be a substantially tubular part with, for example, different external diameters and/or different internal diameters.

The nozzle can, for example, be part of the inner pipe, for example a one-piece integral part of the inner pipe, or be for example releasably or non-releasably connected to the inner pipe, in particular to its first portion.

The nozzle can, for example, be connected preferably releasably to the inner pipe, in particular to its first portion, for example via a fixing mount.

The nozzle can, for example, be attached, preferably releasably, to the inner pipe, in particular to its first portion, for example it may pushed in or on, preferably by means of a plug and/or clamp connection. A screw connection, for example, is also possible.

The nozzle may, for example, expediently form a substantially coaxial and/or output-sided extension of the inner pipe, in particular of the first portion.

The ring element can preferably be attached to the nozzle by press fitting.

The nozzle can preferably be enclosed by the ring element, preferably below its outlet opening for disinfectant.

The nozzle and the inner pipe can preferably extend substantially coaxially and/or form, for example, a common supply channel for the disinfectant, through which the disinfectant can be expediently transported to the plate and/or the bell cup.

The nozzle may have a passage channel for the disinfectant, the passage cross-section of which may, for example, be substantially invariable, may, for example, widen in the direction of flow and/or may, for example, taper in the direction of flow. It is also possible, for example, that the passage cross-section is sectionally invariable, sectionally widens in the direction of flow and/or sectionally tapers in the direction of flow.

The passage channel may, for example, extend substantially axially through the fixing mount of the nozzle.

It is possible that droplets, in particular disinfectant droplets, may form at the nozzle outlet (expediently the outlet opening of the nozzle) if the flow conditions are not optimal, which may run down on the outside of the nozzle. If the flow conditions are not optimal, it is possible, for example, that a fluid jet, in particular a disinfectant fluid jet, does not form directly, which builds up between the nozzle outlet and the underside of the plate. Thereby it is possible that droplets, along the outside of the nozzle and, for example, along the inner pipe, enter in the inside of the housing and/or in a lower area of the apparatus and, for example, cause corrosion.

The ring element is provided in particular so as to prevent or at least delay this.

The bell cup preferably comprises a base, which may be mounted (for example, screwed) on the drive shaft.

The ring element preferably extends at least sectionally in the base of the bell cup. The base of the bell cup can thus, for example, surround the ring element, preferably at a radial distance.

The ring element is preferably disposed outside the housing.

It is possible that the ring element is attached by at least one barb or retaining tooth and preferably the at least one barb or the at least one retaining tooth is disposed on the nozzle.

The at least one barb or the at least one retaining tooth can, in particular, engage with an inner lateral surface of the ring element.

The plate comprises a connecting device, in particular for connecting to the bell cup.

As previously mentioned, the plate preferably forms a deflector plate and/or distributor plate, preferably to transport (expediently by means of its underside) disinfectant discharged from the nozzle to the bell cup, in particular to its inner face.

The plate is preferably rotationally fixed to the connecting device.

The connecting device, in particular its upper side, is preferably expediently spaced from an underside of the plate, for example so as to enable disinfectant discharged from the nozzle to be transported onto the inner face of the bell cup by means of the underside of the plate.

The plate can, for example, be mounted on the connecting device as a separate component, for example, it may be screwed, glued or welded etc. to the connecting device. However, the plate can also be connected directly or indirectly to the connecting device, for example by means of a plug and/or clamp connection. Other connection techniques are also possible.

However, the plate and the connecting device can also form a one-piece integral component, for example.

The nozzle and/or the ring element may, for example, extend in the connecting device, in particular in a through-opening of the connecting device. However, it is also possible, for example, that the ring element does not extend into the connecting device.

It is possible that a first front face of the ring element is substantially exposed towards the plate (expediently long embodiment) or is overlaid (for example spanned) by the connecting device (expediently short embodiment). Alternatively or additionally, a second front face of the ring element can, for example, be mounted on a preferably laterally projecting bearing section of the nozzle.

The ring element can thus be inserted in an expediently long embodiment or expediently short embodiment.

The ring element can, for example, end before an underside of the connecting device (for example, extend to the underside or just before the underside of the connecting device (expediently short embodiment)).

However, the ring element can also extend into an outlet-sided end section of the nozzle (for example, to the nozzle outlet or just before the nozzle outlet (expediently long embodiment)).

As previously mentioned, it is possible that droplets, in particular disinfectant droplets, may form at the nozzle outlet (expediently the outlet opening of the nozzle) if the flow conditions are not optimal, which may run down on the outside of the nozzle. If the flow conditions are not optimal, it is possible, for example, that a fluid jet, especially a disinfectant jet, does not form directly, which builds up between the nozzle outlet and the underside of the plate. Thereby it possible that droplets, along the outside of the nozzle and, for example, along the hollow inner pipe, enter in the inside of the housing and/or in a lower area of the apparatus and, for example, cause corrosion.

As also mentioned previously, the ring element is provided in particular so as to prevent or at least delay this.

However, in order to advantageously reduce or avoid liquid loading of the ring element, the apparatus can comprise, for example, a centrifugal force discharge of liquid, in particular disinfectant and/or rinsing agent.

The connecting device may, for example, have a substantially axial through-opening and/or at least one side channel.

It is possible that liquid, in particular disinfectant and/or rinsing agent, can be fed to the ring element via the through-opening.

The side channel may expediently branch off laterally from the through-opening and/or be configured to laterally discharge liquid running down (for example along the nozzle) by centrifugal force, for example so as to reduce or avoid liquid loading of the ring element.

Liquid that does not enter the side channel despite centrifugal force discharge can be taken in by the ring element so as to be advantageously prevented from penetrating the housing.

It is possible that the nozzle extends expediently at least sectionally in the through-opening and/or the through-opening is delimited internally (for example radially) by the nozzle and/or externally (for example radially) by the connecting device. The through-opening can thus preferably form an annular channel, preferably with different passage cross-sections.

The through-opening may preferably comprise a section (for example, an annular gap) widening (for example, conically) towards the plate, which may, for example, be formed by a conical portion of the connecting device. The through-opening may alternatively or additionally preferably comprise a section (for example, an annular gap) widening (for example, conically) towards the ring element, (which may, for example, be formed by a conical portion of the connecting device.

The through-opening can, for example, have a constriction (for example an annular formed gap, in particular an annular gap).

The constriction can preferably be formed between the section widening towards the plate and the section widening towards the ring element.

For example, the constriction can be delimited (expediently internally) by an offset formed in particular on the nozzle and/or (expediently externally) by an offset formed in particular on the connecting device.

The offset of the nozzle can protrude outwards from the nozzle, for example.

The offset of the connecting device can protrude from the connecting device, for example inwards.

The side channel of the connecting device can, for example, branch off from the through-opening on the side facing the plate and/or the bell cup at least sectionally before the constriction, for example so as to be able to drain off liquid running down (in particular along the nozzle) before the housing and/or before the ring element.

It is possible that the bell cup, preferably its base, has at least one side channel. The side channel of the bell cup can, for example, be configured for lateral discharge of liquid (for example disinfectant and/or rinsing agent) running down (for example along the nozzle) by centrifugal force.

The side channel of the bell cup can, for example, be configured as a through-channel through a wall of the bell cup, in particular as a through-channel through a wall of the base of the bell cup.

During operation of the apparatus, the plate with the connecting device is, in particular, a rotating part, whereas the nozzle and/or the ring element is a static part, for example.

The connecting device can be configured, in particular internally, for example so as to capture liquid (for example droplets and/or disinfectant) running down along the nozzle, preferably substantially at the constriction, and to discharge it by centrifugal force via the side channel of the connecting device and/or the side channel of the bell cup, for example to the outside of the bell cup.

The side channel of the connecting device and the side channel of the bell cup are preferably connected to each other, in particular so as to discharge liquid from the through-opening, preferably by centrifugal force, for example to the outside of the bell cup, in particular when the bell cup, the connecting device and/or the plate rotates. The side channel of the connecting device can preferably open into the side channel of the bell cup.

The centrifugal force occurs in particular during operation of the apparatus, for example when the bell cup, the connecting device and/or the plate rotates.

It is possible that the connecting device comprises, for example, a plurality of side channels and/or the bell cup may comprise, for example, a plurality of side channels.

The ring element and/or the nozzle usually has to be replaced at intervals, therefore a simplified mounting and/or dismounting of the plate is advantageous, in particular without the need to dismount the bell cup. Advantageously, the ring element and/or the nozzle is accessible and expediently replaceable after the plate has been dismounted.

The connecting device may, for example, be provided with an external thread for connection to an internal thread of the bell cup.

On its outer side, preferably facing away from the housing, the nozzle and/or the ring element, the plate may comprise an engagement device for fastening the connecting device to the bell cup and/or for releasing the connecting device from the bell cup. The engagement device serves in particular for tool engagement.

The engagement device can, for example, comprise a projection (for example a hexagon) by means of which, for example, a screw connection between the connecting device and the bell cup can be expediently released and tightened.

Alternatively or additionally, the engagement device may, for example, comprise one or more recesses (for example, holes).

The engagement device can be configured, for example, decentrally or centrally relative to the plate (for example substantially coaxially with the plate, the connecting device, the nozzle and/or the bell cup).

The preferably several recesses can be disposed, for example, in a hole circle, the hole circle being formed, for example, substantially coaxially with the plate, the connecting device, the nozzle and/or the bell cup. The preferably several recesses can thus be configured, for example, decentrally on the plate.

It should be noted that the connecting device can be screwed to the bell cup, for example, in particular onto or into the base of the bell cup. However, it is also possible that the connecting device is expediently inserted into the bell cup in a rotationally fixed manner, in particular into the base of the bell cup, for example by means of a plug and/or clamp connection. Other in particular rotationally fixed connection techniques are also possible.

It should also be noted that centrifugal force means, in particular, a centrifugal force acting radially outwards.

The nozzle and/or the hollow inner pipe is preferably static, in particular non-rotating, during operation of the apparatus.

The bell cup, the connecting device and/or the plate is preferably rotating during operation of the apparatus.

It is possible that the drive shaft, the inner pipe, the nozzle, the ring element and/or the bell cup extend substantially coaxially.

The preferred embodiments of the invention described above may be combined with each other. Other advantageous developments of the invention are disclosed in the dependent claims or result from the following description of preferred embodiments of the invention in conjunction with the attached figures.

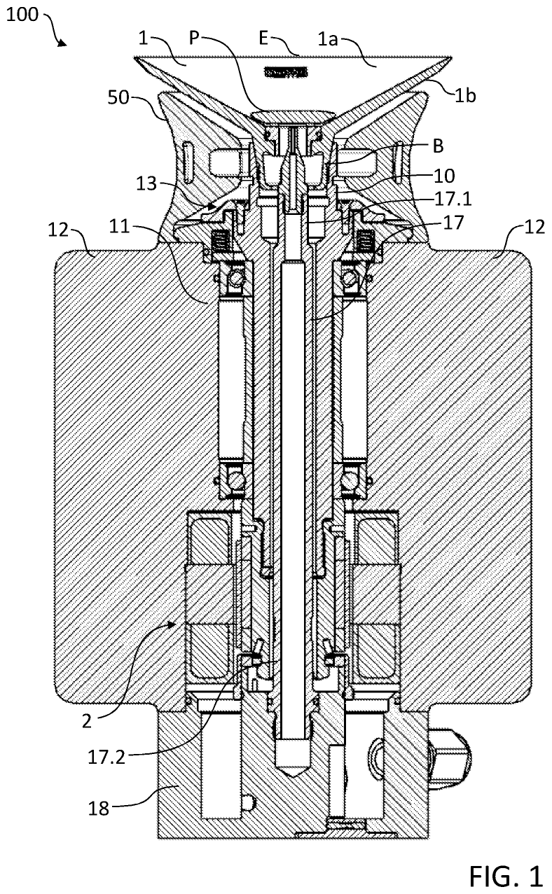

FIG. 1 shows a sectional view of an apparatus according to an embodiment of the invention,

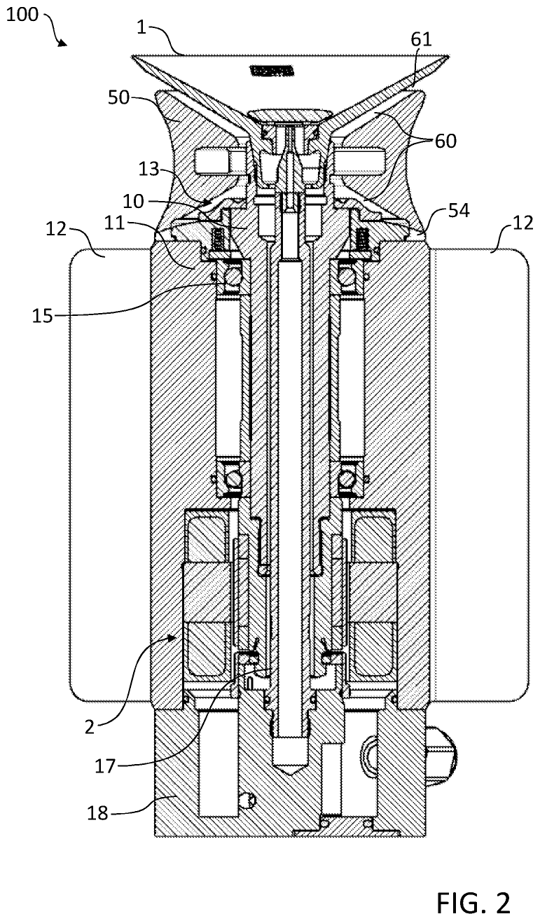

FIG. 2 shows another sectional view of the apparatus,

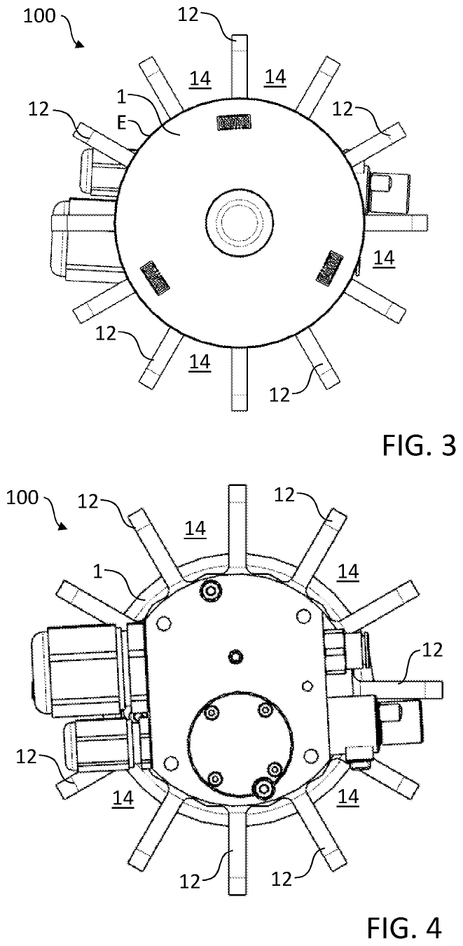

FIG. 3 shows a plan view of the apparatus from above,

FIG. 4 shows a plan view of the apparatus from below,

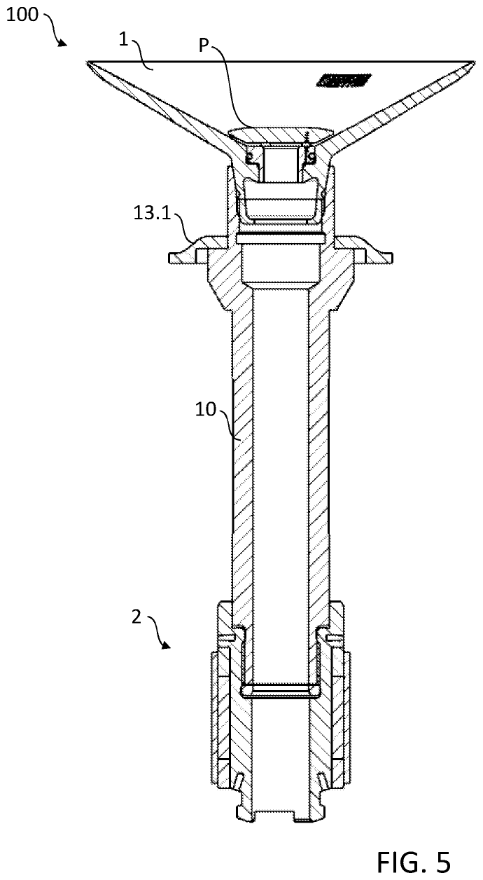

FIG. 5 shows a sectional view of a jointly balanced unit of the apparatus,

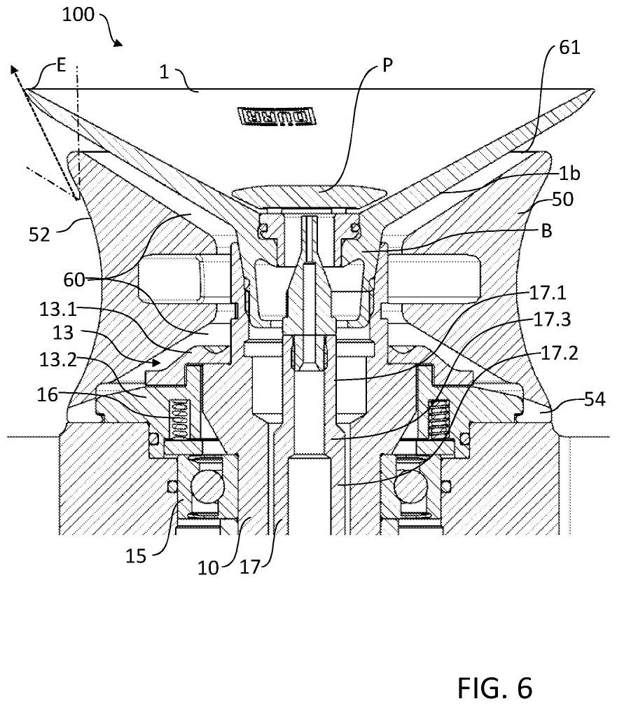

FIG. 6 shows an enlarged sectional view of a part of the apparatus,

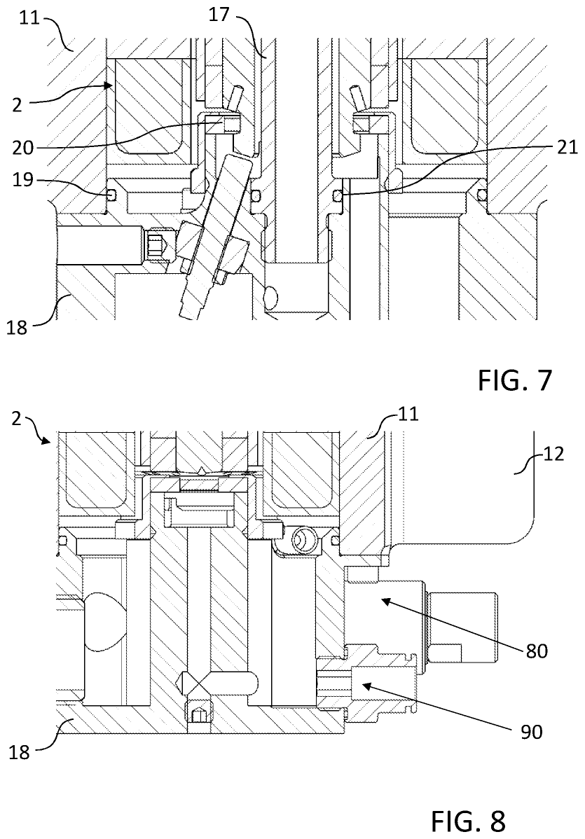

FIGS. 7 and 8 show enlarged sectional views of a part of the apparatus,

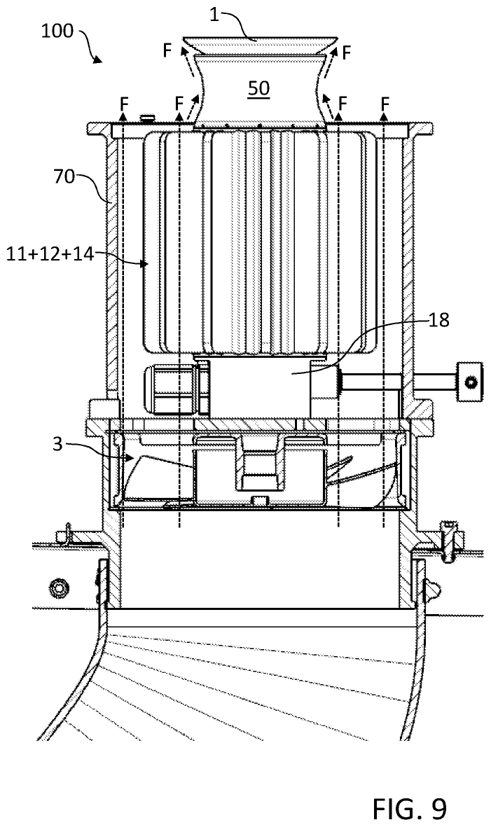

FIG. 9 shows a partial sectional view of the apparatus with, in particular, an enclosure and a blower according to an embodiment of the invention,

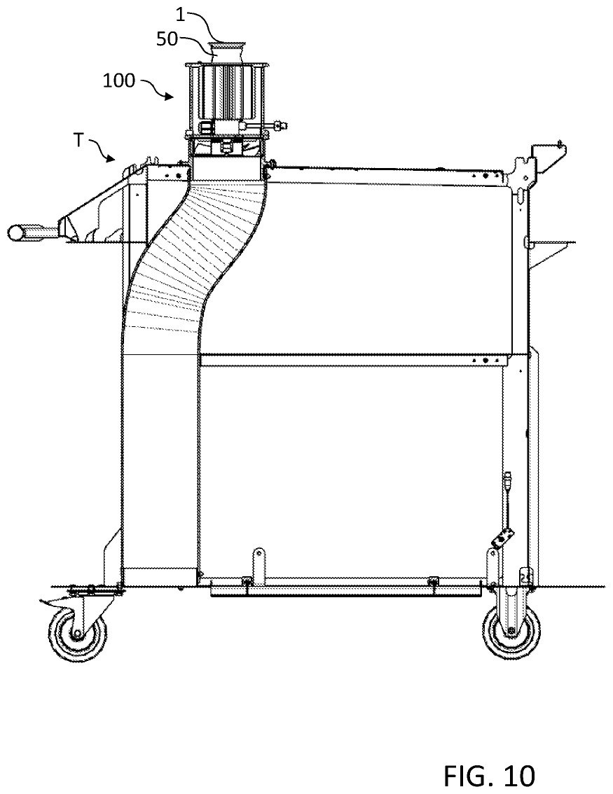

FIG. 10 shows a side view of the structure shown in FIG. 9 with a mobile transportation vehicle according to an embodiment of the invention,

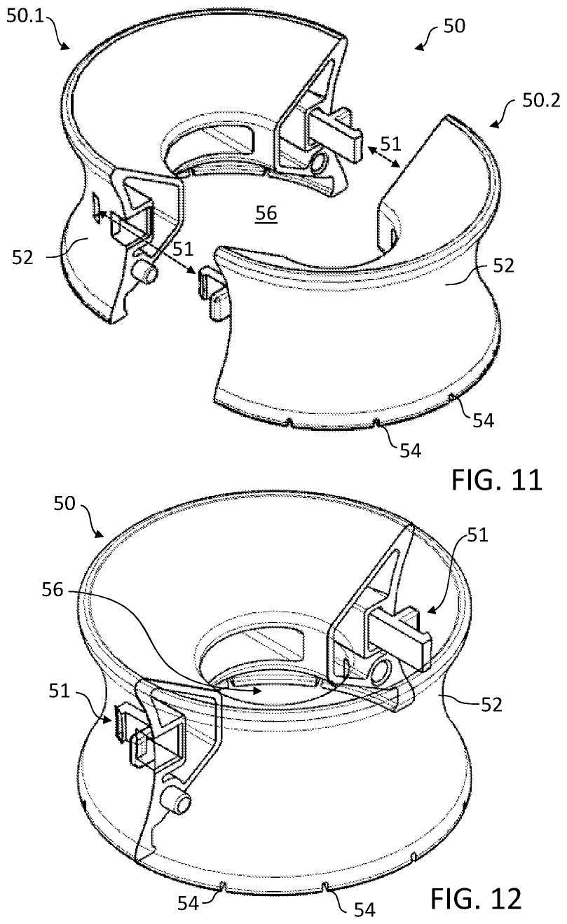

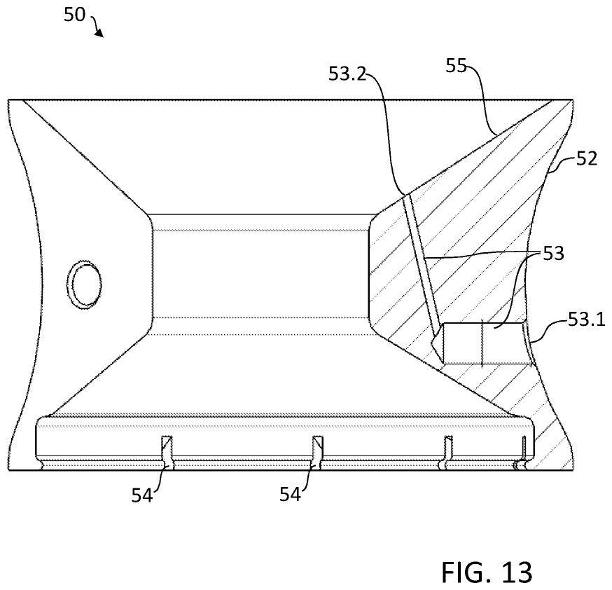

FIGS. 11 to 13 show different views for illustrating a ring structure according to an embodiment of the invention,

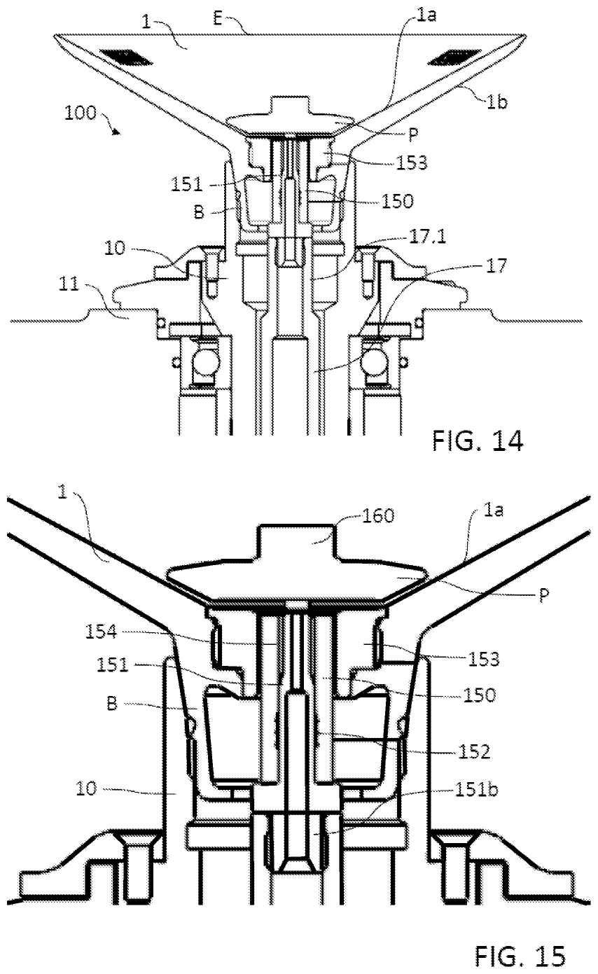

FIG. 14 shows a sectional view of a part of an apparatus according to an embodiment of the invention,

FIG. 15 shows an enlarged sectional view of the apparatus of FIG. 14,

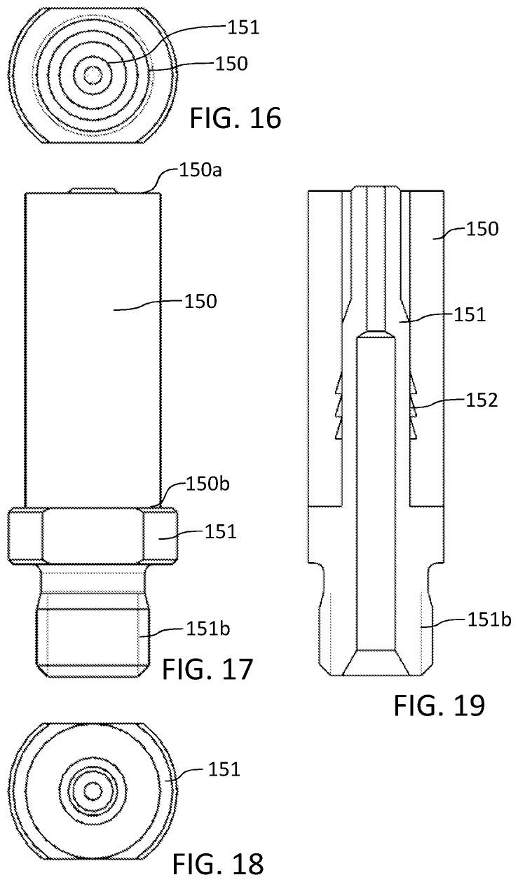

FIG. 16 shows a view from above of a nozzle with a ring element for the apparatus of FIG. 14,

FIG. 17 shows a side view of the nozzle with the ring element,

FIG. 18 shows a view from below of the nozzle with the ring element,

FIG. 19 shows a sectional view of the nozzle with the ring element,

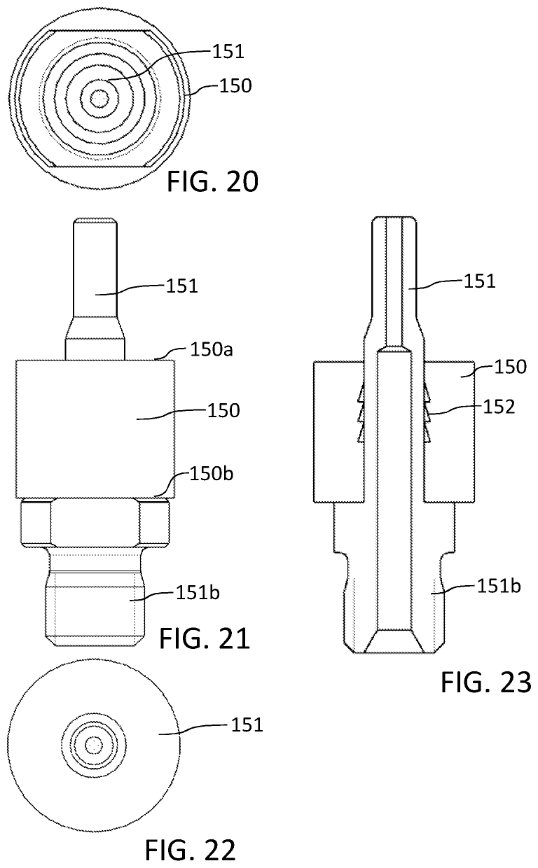

FIG. 20 shows a view from above of a nozzle with a ring element according to another embodiment of the invention,

FIG. 21 shows a side view of the nozzle with the ring element of FIG. 20,

FIG. 22 shows a view from below of the nozzle with the ring element of FIG. 20,

FIG. 23 shows a sectional view of the nozzle with the ring element of FIG. 20,

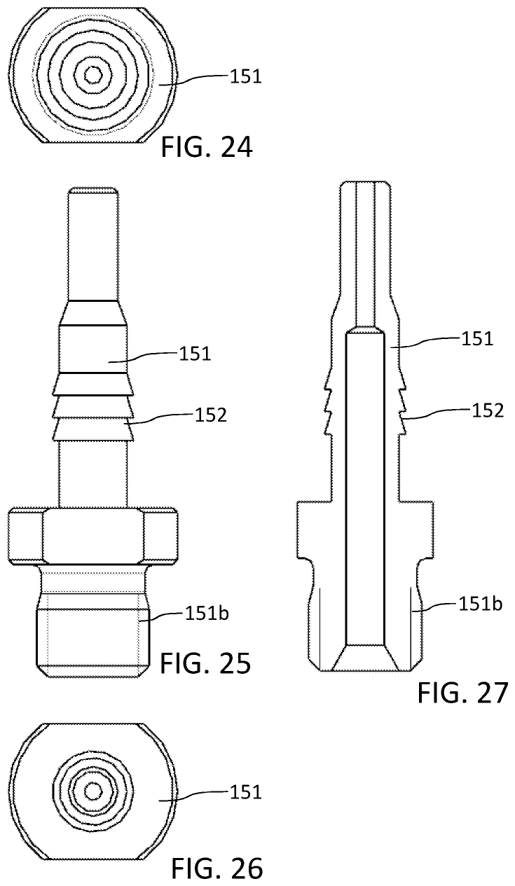

FIG. 24 shows a view from above of a nozzle according to an embodiment of the invention,

FIG. 25 shows a side view of the nozzle of FIG. 24,

FIG. 26 shows a view from below of the nozzle of FIG. 24,

FIG. 27 shows a sectional view of the nozzle of FIG. 24,

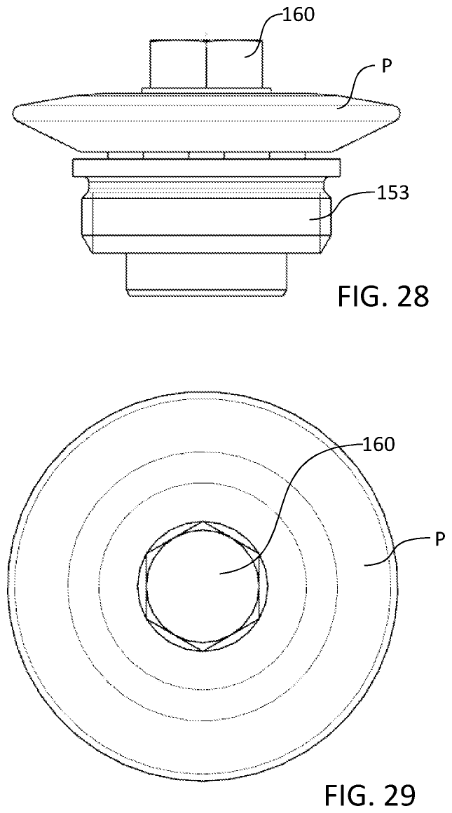

FIG. 28 shows a side view of a plate configured in particular as a deflector plate and/or distributor plate with connecting device according to an embodiment of the invention,

FIG. 29 shows a view from above of the plate with connecting device of FIG. 28,

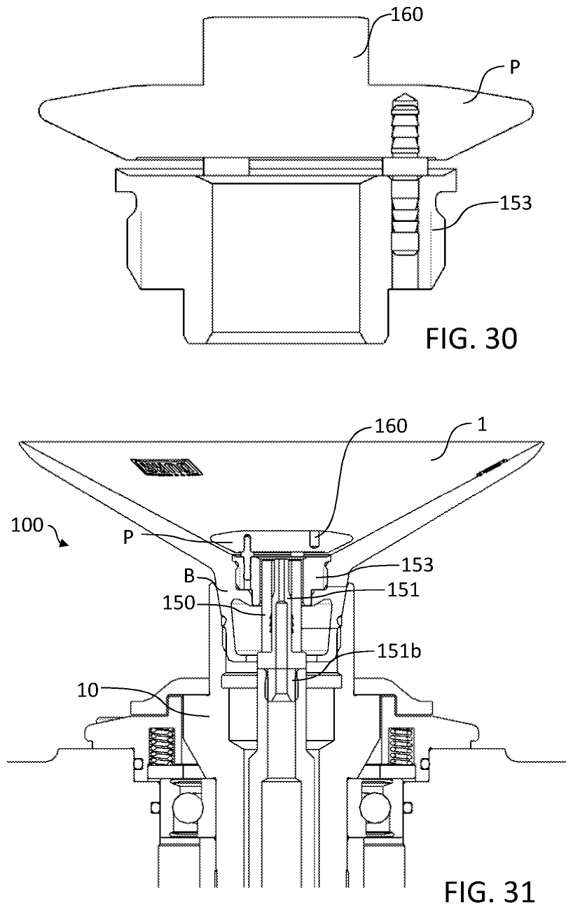

FIG. 30 shows a sectional view of the plate with connecting device of FIG. 28,

FIG. 31 shows a sectional view of a part of an apparatus according to another embodiment of the invention,

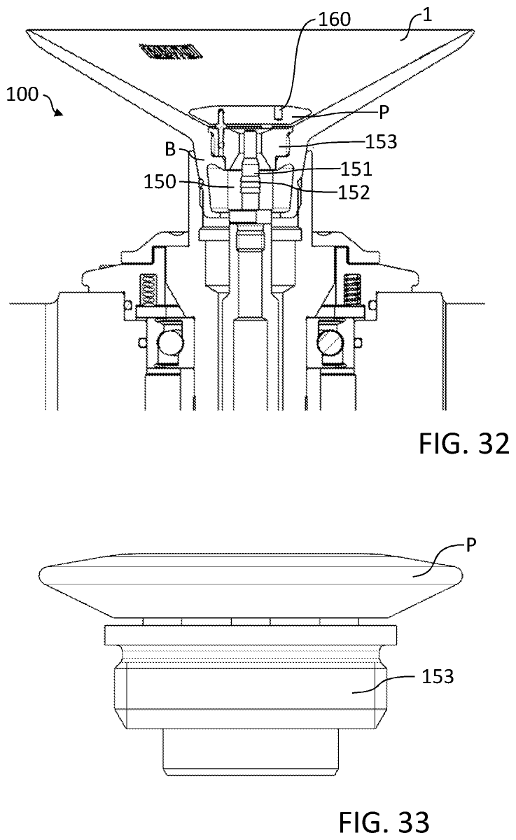

FIG. 32 shows a sectional view of a part of an apparatus according to yet another embodiment of the invention,

FIG. 33 shows a side view of a plate configured in particular as a deflector plate and/or distributor plate with connecting device according to an embodiment of the invention,

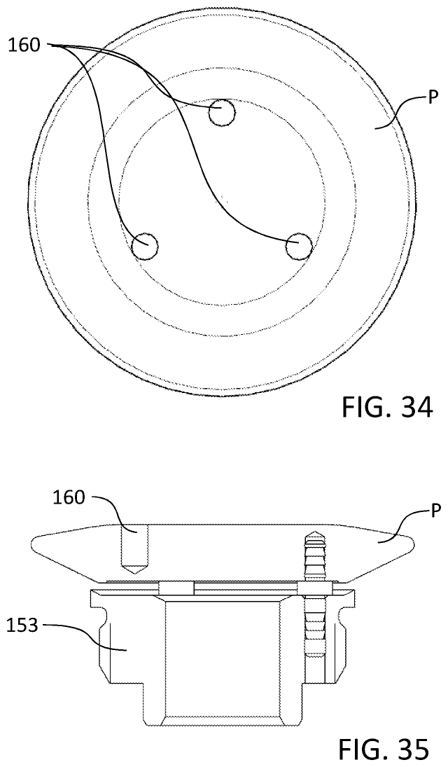

FIG. 34 shows a view from above of the plate of FIG. 33,

FIG. 35 shows a sectional view of the plate of FIG. 33,

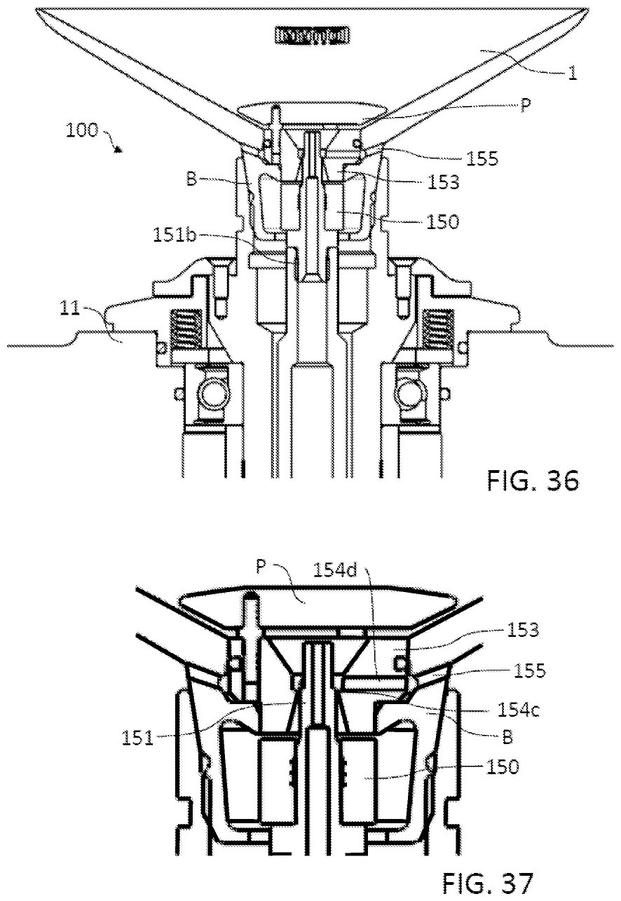

FIG. 36 shows a sectional view of a part of an apparatus according to yet another embodiment of the invention,

FIG. 37 shows an enlarged sectional view of the apparatus of FIG. 36,

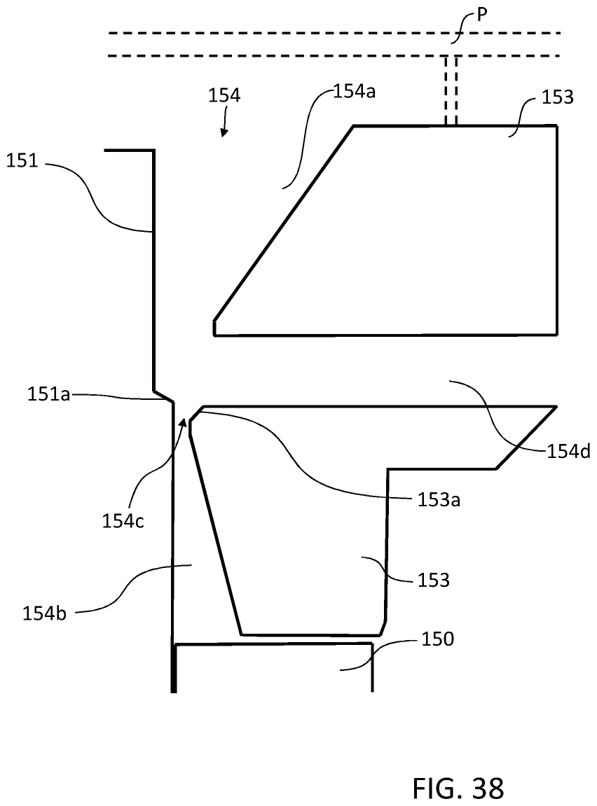

FIG. 38 shows a simplified illustration of a nozzle and a connecting device for the apparatus of FIG. 36,

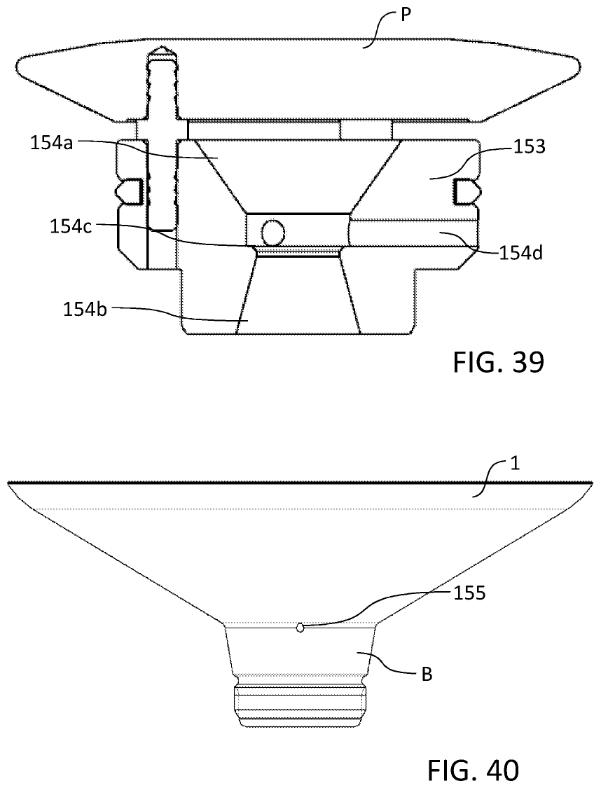

FIG. 39 shows a sectional view of a plate, in particular in the form of a deflector plate and/or distributor plate, with connecting device for the apparatus of FIG. 36,

FIG. 40 shows a side view of a bell cup for the apparatus of FIG. 36,

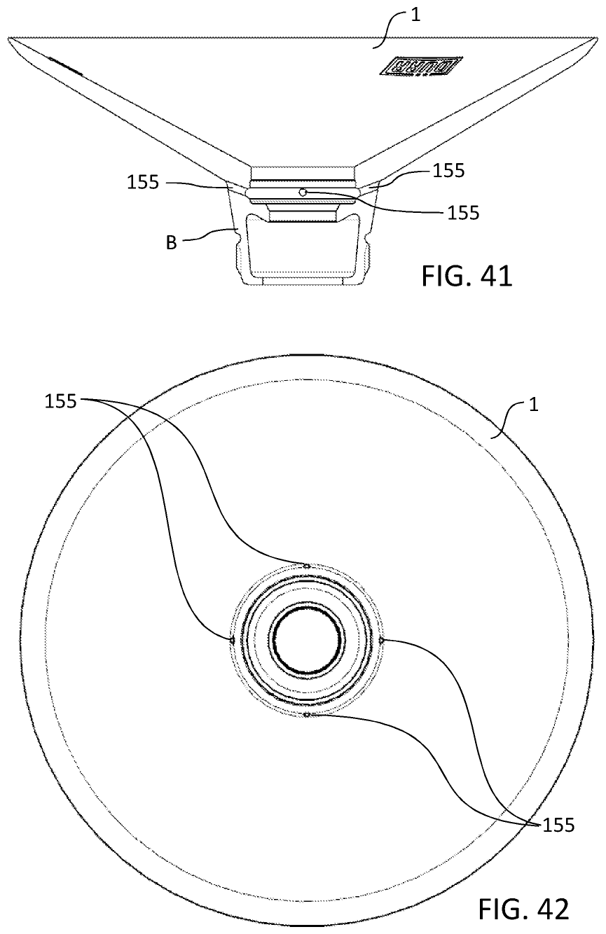

FIG. 41 shows a sectional view of the bell cup of FIG. 40,

FIG. 42 shows a view from below of the bell cup of FIG. 40, and

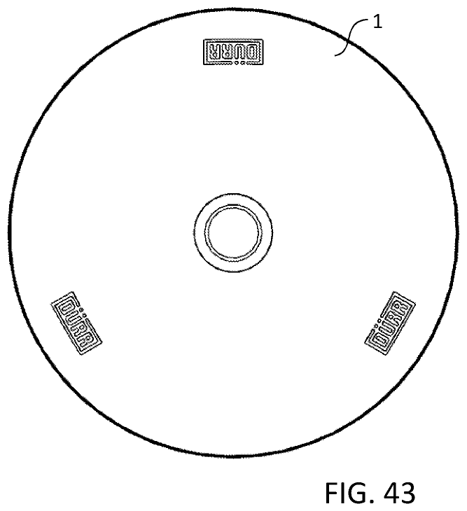

FIG. 43 shows a view from above of the bell cup of FIG. 40.

For the sake of clarity, not all parts are marked with reference signs in all figures.

FIGS. 1 to 6 show different views of an apparatus 100 according to an embodiment of the invention.

The apparatus 100 serves for disinfecting a room, in particular for one or more persons, preferably a dwelling room, storage room, cooling room or treatment room, for example a treatment room of a building, in particular a sickroom, a patient room and/or an operating room. The apparatus 100 may, for example, be partially configured as disclosed in WO2019/238503 A1. The apparatus 100 may also be integrated, for example, into an overall system as described in WO2019/238503 A1.

The apparatus 100 comprises in particular a rotatable, expediently substantially funnel-shaped bell cup 1 for atomising a disinfectant expediently into the room, a hollow drive shaft for driving and in particular rotating of the bell cup 1, and a drive unit 2 (for example, an electric motor) for driving and in particular rotating the drive shaft 10. In the hollow drive shaft 10 there extends an inner pipe 17, which is static and thus non-rotating during operation, for supplying disinfectant. The disinfectant is preferably a hypochlorous and usually temperature-sensitive acid (HOCl).

The bell cup 1 comprises an inner face 1a, an outer face 1b, a base section B and a circumferential spray edge E for spraying the disinfectant.

The bell cup 1 interacts with a disc-shaped plate P, configured as a deflector plate or distributor plate, which expediently rotates during operation. The bell cup 1, the drive shaft 10, the inner pipe 17 and the plate P are substantially disposed coaxially with one another. In operation, the disinfectant fed through the drive shaft 10 and the inner pipe 17 meets the rotating plate P and is thereby distributed through centrifugal force onto the inner face 1a of the rotating bell cup 1, from where the disinfectant is led to the annular spray edge E, from where it is sprayed in an atomised form into the room. In operation, the bell cup 1 can have a rotational speed of, for example, at least 30,000 revolutions per minute and preferably have an atomising capacity of at least 50 millilitres per minute and/or at least 0.3 cubic metres per minute.

Bell cups and their mode of operation, in particular with a rotating plate, are common in the technical field of painting motor vehicle bodies by means of rotary atomisers, so that reference can be made thereto for details.

A housing 11 is provided, in which, in particular, the drive unit 2 is accommodated. Also, in particular the drive shaft 10 and thus also the inner pipe 17 can be accommodated at least sectionally in the housing 11.

Relatively high temperatures can occur during operation, especially in the housing 11. There may be a risk that the high temperatures damage or negatively affect the disinfectant.

The housing 11 is therefore provided with a plurality of cooling ribs 12 (for example, heat ribs or cooling fins) surface enlargement to thus can act as an effective heat exchanger. In particular, this allows heat to be effectively dissipated from the interior of the housing 11 to the outside, for example so as to be able to maintain temperatures inside the housing 11 at a level suitable for the disinfectant in particular.

The cooling ribs 12 expediently protrude radially laterally outwards from the housing 11, extend parallel to the drive shaft 10 and are uniformly spaced from each other in the circumferential direction of the housing 11, for example.

Thereby, gas guiding channels 14 can be formed between the cooling ribs 12, which can be used to guide a gas flow F generated by a blower 3 (for example, FIG. 9). The blower 3 is designed, for example, as an axial fan and disposed upstream of, in particular underneath, the housing 11.

The blower 3 and the cooling ribs 12 and thus the gas guiding channels 14 are configured such that the gas flow F in the gas guiding channels 14 is guided along the cooling ribs 12 towards the bell cup 1 and/or towards the atomised disinfectant in order to distribute the atomised disinfectant in the room.

The blower 3 and the gas flow F can thus serve, on the one hand, to distribute the atomised disinfectant and, on the other hand, to effectively dissipate heat from the cooling ribs 12.

The housing 11 and/or the cooling ribs 12 may have a recess for receiving a part of the drive unit 2, wherein the cooling ribs 12 can sectionally extend laterally outside along the drive unit 2.

A particular challenge is to protect sensitive components, particularly inside the housing 11, from the hypochlorous acid, but also from other, possibly less corrosive liquids such as rinsing agents, room humidity, etc.

In order to prevent penetration of the hypochlorous acid in particular during regular operation, one challenge is to seal particularly the head side of the housing 11 between stator (in particular the housing 11) and rotor (in particular the rotating drive shaft 10). Statically contacting seals have proved negative in this respect.

Therefore, the drive shaft 10 is provided with a labyrinth seal 13, preferably in order to prevent a head-sided unwanted penetration of liquid (especially disinfectant, rinsing agent, etc.) into the housing 11.

The labyrinth seal 13 comprises a first, in particular annular, section 13.1 and a second, in particular annular, section 13.2.

The first section 13.1 is associated with the drive shaft 10 and is co-rotatable with the drive shaft 10.

The second section 13.2 is associated with the housing 11 and is static so that it does not rotate during operation.

An intermediate gap is disposed between the first section 13.1 and the second section 13.2 so that the first section 13.1 and the second section 13.2 preferably do not touch each other. The labyrinth seal 13 is thus preferably configured as a contactless labyrinth seal 13.

A particular feature is that the intermediate gap is in particular so thin that during operation an air barrier is formed in the intermediate gap through rotation of the first section 13.1 and only the air barrier acts as a seal against liquid.

Thus, it is possible that the labyrinth seal 13 is inactive when the drive shaft 10 is not rotating and thus when the first section 13.1 is not rotating, and therefore liquid could enter the interior of the housing 11 via the intermediate gap.

The apparatus 100 may therefore have a control device which is configured to allow the drive shaft 10, and thus in particular the first section 13.1 of the labyrinth seal 13, to post-rotate for a predefined period of time after an atomisation process.

In this way, the labyrinth seal 13 can be kept in an activated state, in particular until no liquid or only a negligible amount of liquid (for example, disinfectant and/or rinsing agent) is present on the labyrinth seal 13, in particular until liquid that may have accumulated in the expediently annular-gap-shaped intermediate space 60 has been able to drain off.

In the housing 11 also a bearing 15 (for example, a roller bearing, etc.) for, in particular, rotatable mounting of the drive shaft 10 is accommodated.

An elastic loading element 16 such as, for example, a spring, a rubber, etc. may be disposed between the second section 13.2 and the bearing 15 in order to act on the bearing 15. The loading element 16 serves in particular for pre-tension of the bearing 15 and/or for tolerance compensation of the bearing 15.

In operation, the disinfectant is fed to the bell cup 1 via the non-rotating inner pipe 17 and preferably the plate P. The inner pipe 17 extends inside the drive shaft 10, which rotates in operation, so that it may be subjected thereby to relatively strong vibrations.

Therefore, the inner pipe 17 may be divided into a first portion 17.1 and a second portion 17.2. The first portion 17.1 may expediently face the bell cup 1 and have, for example, an outlet opening for disinfectant.

The outlet opening may, for example, comprise a specially shaped nozzle for increasing pressure with a defined outlet diameter of preferably 0.5 mm<=d<=0.9 mm.

The second portion 17.2 may, for example, have an entrance opening for the disinfectant, run between the drive unit 2 and/or be carried in a mount 18 for the housing 11.

It has been found to be advantageous if the second portion 17.2 has a greater external diameter than the first portion 17.1, has a greater wall thickness than the first portion 17.1 and, in particular, also has a greater passage cross-section for the disinfectant than the first portion 17.1. A stepped offset 17.3 between the first portion 17.1 and the second portion 17.2 has also been found to be advantageous.

The mount 18 can also form a supply block in which at least one supply line 80 for liquid (for example, FIG. 8), preferably the disinfectant and/or rinsing agent, can run, at least one control line for the drive unit 2 can run, at least one drive line (for example, a current line, compressed air line, etc.) for the drive unit 2 can run, etc.

The apparatus 100 also comprises a ring structure 50 with an outer face 52 which is in particular concave and/or extends laterally outwards, preferably in order to guide further the gas flow F generated by the blower 3 laterally outwards, as shown schematically in FIG. 6. In FIG. 6, the outer face 52 is substantially directed towards the spray edge E of the bell cup 1, wherein deviations of less than +/−30° are possible, as schematically indicated by the dash-dot lines in FIG. 6.

The ring structure 50 serves for encasing or covering of the base B of the bell cup 1, a part of the outer face 1b of the bell cup 1 and/or the labyrinth seal 13, whereby in particular a noise reduction can be achieved. The ring structure 50 can thus form a noise reduction skirt.

The ring structure 50 externally delimits a particularly annular-gap-shaped intermediate space 60, which can be delimited internally, in particular by the base B of the bell cup 1, a part of the outer face 1b of the bell cup 1 and/or the labyrinth seal 13.

The intermediate space 60 has a, for example, annular inlet opening 61, so that liquid such as disinfectant, rinsing agent etc. can enter the intermediate space 60 via the inlet opening 61.

Therefore, one or more preferably slit-shaped exit openings 54 are provided through which the liquid can flow out of the intermediate space 60, preferably gravity-based alone. The exit openings 54 are expediently disposed in a lower base section of the ring structure 50 and are slit-shaped.

FIG. 5 shows a sectional view of a preferably jointly balanced unit, wherein the unit may comprise the bell cup 1, the rotatable part of the drive unit 2, the first section 13.1 of the labyrinth seal 13 and the plate P.

FIGS. 7 and 8 show enlarged sectional views of a part of the apparatus 100, and in particular of a lower part of the apparatus 100 with the mount 18 for the housing 11.

Due to, for example, negative pressure conditions in the housing 11, it is possible that HOCl-laden air can flow into the housing 11, so that liquid, moisture, etc. could also enter into the housing 11 from below.

An O-ring seal 19 is disposed between the mount 18 and the housing 11, in particular to expediently seal the housing 11 from below and thus in particular mount-sided. Primary objective is to ensure, in particular, that no liquid can penetrate to the drive unit 2. An O-ring seal 21 is disposed between the mount 18 and the inner pipe 17, in particular to expediently seal the housing 11 from below and thus in particular mount-sided.

The apparatus 100 may also comprise an expedient annular brush seal 20, and preferably an anti-static brush seal, in particular a carbon fibre brush seal, to seal the housing 11.

The brush seal 20 is preferably statically attached to the mount 18.

The brush seal 20 can be disposed on the rotatable part (rotor) of the drive unit 2, in particular so as to expediently seal the housing 11 from below, in particular in such a way that no liquid can penetrate into the drive unit 2.

The seals described before are advantageous for sealing the housing 11, in particular from below and thus mount-sided. However, the problem here is that liquid could accumulate undesirably in the housing 11 because it cannot drain downwards out of the housing 11.

If nevertheless, for example, liquid penetrates into the area of the drive unit 2, a leakage occurs at the inner pipe 17 and/or there is a backflow of liquid from the nozzle opening or outlet opening, the in particular highly corrosive liquid can be drained off through at least one leakage outlet 90 (for example, FIG. 8).

The leakage outlet 90 is preferably disposed in the mount 18 and/or floor-sided in the housing 11.

FIG. 9 shows a partial sectional view of the apparatus 100 with, in particular, an enclosure 70, the blower 3 for generating the gas flow F and a suction line for the blower 3, wherein FIG. 10 shows a side view of the assembly shown in FIG. 9 with a mobile transportation vehicle T.

FIGS. 9 and 10 show in particular that the housing 11 with the cooling ribs 12 is surrounded by the (for example, cylindrical) enclosure 70, wherein the gas flow F generated by the blower 3 can be guided through the enclosure 70. The enclosure 70 is preferably laterally spaced from the cooling ribs 12. The bell cup 1 and the ring structure 50 are preferably disposed at least sectionally outside the enclosure 70.

FIGS. 11 to 13 show different views for illustrating a particularly advantageous ring structure 50.

A special feature is that the ring structure 50 is, for example, openable and thus expediently dismountable and/or is composed of several sections 50.1, 50.2 (for example, half shells) releasably mountable to each other so as to be able to remove the ring structure 50 from the apparatus 100 and, if necessary, reattach it.

The ring structure 50 and in particular the sections 50.1 and 50.2 can be openable and/or closable, for example, by means of at least one latch, plug and/or clamp connection 51.

Another special feature is also that the ring structure 50 may have, for example, a rinsing agent channel 53 for connecting to a rinsing agent line for supplying rinsing agent. The rinsing agent channel 53 serves to rinse the intermediate space 60.

The ring structure 50 may comprise an expediently central interior space 56 and a side wall construction, wherein the side wall construction can run annularly around the interior space 56.

The rinsing agent channel 53 can, for example, include an inlet hole 53.1 and at least one outlet hole 53.2. The rinsing agent channel 53 is integrated into the side wall construction of the ring structure 50 and extends through the side wall construction of the ring structure 50.

The inlet hole 53.1 is expediently disposed in the outer face 52 and may comprise a connecting device for connecting to the rinsing agent line. The connecting device can, for example, comprise a thread or another suitable, preferably releasable connection structure.

The at least one outlet hole 53.2 is expediently disposed in an inner face 55 of the ring structure 50 and is directed in particular towards the bell cup 1, in particular towards the outer face 1b of the bell cup 1 facing the ring structure 50. The at least one outlet hole 53.2 may expediently open into the intermediate space 60.

If the intermediate space 60 is rinsed via the rinsing agent channel 53, rinsing agent may accumulate in the intermediate space 60, which can advantageously drain off via the exit openings 54, although this takes a certain amount of time. Disinfectant can also enter the intermediate space 60 via the inlet opening 61, for example by insertion through edge separation during the atomisation process.

A control device of the apparatus 100 may therefore be configured to allow the drive shaft 10, and thus the first section 13.1, to rotate until and therewith to keep the labyrinth seal 13 in an activated state until the disinfectant and/or the rinsing agent has at least substantially drained from the intermediate space 60. For this purpose, a post-rotation after the atomisation and/or rinsing process can be carried out.

It should also be noted that, for example, the disinfectant disclosed in US 2014/0119992 A1 can be used as disinfectant and/or other suitable disinfectants such as hypochlorous acid (HOCl), for example, in order to eliminate, in particular, bacteria, viruses, germs and/or fungi etc. The disinfectant is, in particular, a liquid disinfectant.

The preferred embodiments of the invention described by reference to the figures are partly identical, with similar or identical parts having the same reference signs and, to explain these, reference may also made to the description of the other embodiments in order to avoid repetition. For presentation purposes, not all parts are marked with reference signs in all figures.

FIG. 14 shows a sectional view of a part of an apparatus 100 according to an embodiment of the invention, wherein FIG. 15 shows an enlarged sectional view of the apparatus 100 in FIG. 14.

The apparatus 100 serves for disinfecting at least one room and comprises, in particular, a rotatable bell cup 1 for atomising a disinfectant, a hollow drive shaft 10 for driving the bell cup 1 and, for example, a housing 11 in which a drive unit for driving the drive shaft 10 and preferably the drive shaft 10 may be accommodated. The apparatus 100 may optionally comprise, for example, cooling ribs 12 and/or a labyrinth seal 13 as disclosed herein.

The bell cup 1 has in its centre a rotatably mounted plate P with a connecting device 153.

The plate P is configured as a plate P that rotates with the bell cup 1 and in particular as a deflector plate and/or distributor plate.

The plate P is expediently rotationally fixed to the connecting device 153.

The connecting device 153 serves in particular as an expediently rotationally fixed connection with the bell cup 1, so that the plate P is rotatable together with the bell cup 1. The plate P can, for example, be mounted on the connecting device 153 as a separate component, for example, it may be screwed, glued or welded to the connecting device 153. However, the plate P can also be connected, for example directly or indirectly, by means of a plug and/or clamp connection to the connecting device 153, in particular in a rotationally fixed manner. However, the plate P and the connecting device 153 may also be formed in one-piece integrally, for example. The plate P may expediently comprise on its outer side an engagement device 160 (for example, for tool engagement, preferably a hexagon) for fastening the connecting device 153 to the bell cup 1 and/or for releasing the connecting device 153 from the bell cup 1. The nozzle 151 may extend in a through-opening 154 of the connecting device 153.

The bell cup 1 comprises a base B, which may expediently be mounted on the drive shaft 10.

The apparatus 100 comprises an in particular tubular nozzle 151, having, for example, different external and/or internal diameters, with an outlet opening for discharging disinfectant expediently onto the plate P, in particular its underside. The disinfectant can be transported via the plate P to the inner face 1a of the bell cup 1.

The nozzle 151 is preferably releasably connected to the inner pipe 17, and in particular to the first portion 17.1 of the inner pipe 17. For this purpose, the nozzle 151 may comprise, for example, a fixing mount 151b, which may be configured to be plugged or screwed onto or into the first portion 17.1, for example. The nozzle 151 may, for example, form a substantially coaxial extension of the first portion 17.1 and preferably form a common supply channel for the disinfectant with the inner pipe 17.

The apparatus 100 comprises a ring element 150. The ring element 150 comprises a material (for example, a non-woven fibrous structure, a foamed plastic and/or a felt) with a capacity/material property that allows in particular high liquid intake. Also a combination of different materials is possible for the ring element 150.

It is possible that disinfectant droplets may form at the outlet opening of the nozzle 151 if the flow conditions are not optimal, which may run down on the outside of the nozzle 151, for example. If the flow conditions are not optimal, it is possible, for example, that a disinfectant fluid jet does not form directly, which builds up between the outlet opening of the nozzle 151 and the underside of the plate P. As a result, it is possible that droplets or liquid in general can, along the outside of the nozzle 151 and, for example, along the hollow inner pipe 17, enter into the interior of the housing 11 and/or a lower area of the apparatus 100 and cause corrosion, for example.

The ring element 150 is provided in particular so as to prevent or at least delay this.

The ring element 150 extends, in particular, in the base B of the bell cup 1 and inside the connecting device 153. The ring element 150 is attached to the nozzle 151, for example by press fitting, and preferably surrounds the nozzle 151 substantially by 360°. The ring element 150 is attached, in particular, by one or more barbs or retaining teeth 152 disposed on the nozzle 151. The ring element 150 extends into an outlet-sided end section of the nozzle 151 (for example, up to the nozzle outlet or just before the nozzle outlet (long embodiment)).

The ring element 150 and the nozzle 151 are static, particularly in operation.

FIGS. 16 to 19 show different views of the nozzle 151 and the ring element 150 of the apparatus 100 of FIG. 14.

The ring element 150 comprises a first front face 150a and a second front face 150b.

The first front face 150a is preferably substantially exposed towards the plate P. The second front face 150b is preferably mounted on a bearing section of the nozzle 151, in particular placed on the bearing section.

The plate P is preferably releasably by means of a suitable tool without dismounting the bell cup 1, so that the underlying nozzle 151 and the underlying ring element 150 are accessible and preferably replaceable.

FIGS. 20 to 23 show different views of a nozzle 151 and a ring element 150 according to another embodiment of the invention.

The ring element 150 comprises a first front face 150a and a front face 150b.

The first front face 150a is provided so as to be overlaid by a connecting device 153. The second front face 150b is preferably mounted on a bearing section of the nozzle 151, in particular placed on the bearing section. The embodiment shown in FIGS. 20 to 23 is preferably provided for an apparatus 100 such as that shown, for example, in FIG. 32 or FIG. 36, in which the ring element 150 ends for example before an underside of the connecting device 153 (for example, extends only up to the underside or just before the underside of the connecting device 153 (short embodiment)).

FIGS. 24 to 27 show different views of a nozzle 151 according to an embodiment of the invention. The nozzle 151 is a substantially tubular component with, for example, different external diameters and/or different internal diameters. The nozzle 151 is preferably configured to be expediently connected substantially coaxially with the inner pipe 17, for example via the fixing mount 151b, which can be configured to be plugged or screwed onto or into the inner pipe 17, in particular its first portion 17.1.

FIGS. 28 to 30 show different views of plate P with a connecting device 153 according to an embodiment of the invention. The plate P and the connecting device 153 are connected to each other preferably in a rotationally fixed manner by means of a plug and/or clamp connection, although other connection techniques (for example, gluing, welding or screwing connections) are also possible. A one-piece integral configuration of the plate P and the connecting device 153 is also possible.

The connecting device 153 comprises an external thread for connection to an internal thread of the bell cup 1, wherein the internal thread can expediently be provided on or in the base B of the bell cup 1. However, embodiments are also possible in which the connecting device 153 is not screwed to the bell cup 1, but is connected to the bell cup 1 in a rotationally fixed manner, for example by means of a plug and/or clamp connection.

An upper side of the connecting device 153 is preferably spaced from an underside of the plate P, so that disinfectant discharged from the nozzle 151 can be transported onto the inner face 1a of the bell cup 1 by the underside of the plate P.

In particular, the ring element 150 and, for example, also the nozzle 151 usually have to be replaced at intervals, therefore a simplified mounting and/or dismounting of the plate P is advantageous, in particular without the need of dismounting the bell cup 1.

The plate P therefore comprises, on its outer side, an engagement device 160 for fastening the connecting device 153 to the bell cup 1 and/or for releasing the connecting device 153 from the bell cup 1. The engagement device 160 is configured, for example, substantially coaxially with the plate P, the connecting device 153, the nozzle 151 and/or the bell cup 1 and, for example, configured as hexagon. In particular, the engagement device 160 is provided on top of the plate P in order to allow, for a change of a ring element 150 and/or a nozzle 151, an advantageously quick and easy mounting and dismounting of the plate P and the connecting device 153.

FIG. 31 shows a sectional view of a part of an apparatus 100 according to another embodiment of the invention. The embodiment corresponds substantially to the embodiment of FIG. 14, wherein a different plate/connecting device construction (P and 153) is used. The embodiment of FIG. 31 illustrates, in particular, an example of a ring element 150 whose front side 150a is exposed towards the plate P.

FIG. 32 shows a sectional view of a part of an apparatus 100 according to yet another embodiment of the invention. The embodiment uses, in particular, a nozzle/ring element construction (151 and 150) as described with reference to FIGS. 20 to 23. The embodiment of FIG. 32 illustrates, in particular, an example of a ring element 150 whose front side 150a can be overlaid, at least sectionally, by a connecting device 153.

FIGS. 33 to 35 show different views of a plate P with a connecting device 153 according to an embodiment of the invention. The plate P comprises, on its outer side, an engagement device 160 for fastening the connecting device 153 to the bell cup 1 and/or for releasing the connecting device 153 from the bell cup 1. In particular, the engagement device 160 is provided on top of the plate P in order to allow, for a change of a ring element 150 and/or a nozzle 151, an advantageously quick and easy mounting and dismounting of the plate P and the connecting device 153.

The engagement device 160 comprises, in particular, a plurality of recesses disposed in a hole circle, wherein the hole circle can be formed, for example, substantially coaxially with the plate P, the connecting device 153, the nozzle 151 and/or the bell cup 1. The recesses, on the other hand, are configured decentrally on the plate P.

FIG. 36 shows a sectional view of a part of an apparatus 100 according to an embodiment of the invention, wherein FIG. 37 shows an enlarged sectional view of the apparatus 100. FIG. 38 illustrates, in particular, a nozzle 151 and a connecting device 153 together with an associated plate P for the apparatus 100, wherein FIG. 39 shows a sectional view of the plate P with the connecting device 153. FIGS. 40 to 43 show different views of a bell cup 1 for the apparatus 100.

The connecting device 153 comprises a preferably axial through-opening 154 and/or at least one side channel 154d. The nozzle 151 extends in the through-opening 154 so that the through-opening 154 is delimited internally by the nozzle 151 and externally by the connecting device 153.

The through-opening 154 comprises a section 154a widening towards the plate P, expediently formed by a conical portion of the connecting device 153.

The through-opening 154 comprises a section 154b widening towards the ring element 150, expediently formed by a conical portion of the connecting device 153.

The through-opening 154 also comprises a constriction 154c, configured, for example, as an annular gap, which can be expediently formed between the section 154a and the section 154b.

The constriction 154c may in particular be delimited internally by an offset 151a formed on the nozzle 151 and, in particular, externally by an offset 153a formed on the connecting device 153.

The side channel 154d branches off laterally from the through-opening 154 and serves in particular to laterally discharge liquid, in particular disinfectant and/or rinsing agent, by radially acting centrifugal force.

The side channel 154d of the connecting device 153 branches off from the through-opening 154 on the side facing the plate P at least sectionally before the constriction 154c.

The bell cup 1 may also have at least one side channel 155, which may, for example, form an extension of the side channel 154d.

The side channel 154d of the connecting device 153 and the side channel 155 of the bell cup 1 are preferably connected to each other so as to be able to discharge liquid from the through-opening 154 by centrifugal force via the side channel 154d of the connecting device 153 and the side channel 155 of the bell cup 1, for example to the outside of the bell cup 1.

The connecting device 153 rotates during operation of the apparatus 100. The nozzle 151, on the other hand, is non-rotating, in particular static, during operation of the apparatus 100.

The rotating connecting device 153 is configured, in particular, to capture liquid (for example, a droplet and/or disinfectant) running down, for example, along the non-rotating nozzle 151, preferably substantially at or shortly before the constriction 154c, and to discharge it by centrifugal force via the side channel 154d of the connecting device 153 and/or the side channel 155 of the bell cup 1, for example to the outside of the bell cup 1.

A droplet running down can thus entrained by the connecting device 153 of the plate P substantially at the constriction 154c and be discharged laterally by radially acting centrifugal forces.

Centrifugal force is generated during operation of the apparatus 100 when the rotatable parts rotate, for example, the bell cup 1, the plate P with the connecting device 153 and/or the drive shaft 10. The nozzle 151 and, for example, the inner pipe 17, on the other hand, do not rotate during operation of the apparatus 100, as previously mentioned.

Liquid that does not enter the side channel 154d despite centrifugal discharge can be taken in by the ring element 150 and thus be prevented from entering the housing 11, for example.

The invention is not limited to the preferred embodiments described above. Rather, a large number of variants and modifications are possible, which also make use of the inventive concept and thus fall within the scope of protection. Furthermore, the invention also claims protection for the subject matter and features of the dependent claims independently of the features and claims referred to.

### LIST OF REFERENCES

1 Bell cup
1a Inner face of the bell cup
1b Outer face of the bell cup
B Base of the bell cup, in particular mounting section
E Spray edge of the bell cup
P Plate, in particular distributor/deflector plate
2 Drive unit, preferably motor
3 Blower, in particular axial fan
F Gas flow, preferably air flow
10 Hollow drive shaft
11 Housing, preferably drive unit housing
12 Cooling ribs
13 Labyrinth seal
13.1 First section
13.2 Second section
14 Gas guiding channels
15 Bearing
16 Loading element
17 Hollow inner pipe
17.1 First portion
17.2 Second portion
17.3 Stepped offset
18 Mount for housing
19 O-ring seal
20 Brush seal
21 O-ring seal
50 Ring structure
50.1 Section
50.2 Section
51 Latch, plug and/or clamp connection
52 Outer face, in particular, outer lateral surface
53 Rinsing agent channel
53.1 Inlet hole
53.2 Outlet hole
54 Preferably slit-shaped exit opening, in particular, run-off opening
55 Inner face, in particular, inner lateral surface
56 Interior space
60 Intermediate space
61 Inlet opening
70 Enclosure
80 Supply line
90 Leakage outlet
T Transportation vehicle
100 Apparatus
150 Ring element, in particular for liquid intake
150a Front face of ring element
150b Front face of ring element
151 Nozzle
151a Offset of the nozzle
151b Fixing mount of the nozzle
152 Barb or tooth
153 Connecting device
153a Offset of the connecting device
154 Through-opening of the connecting device
154a Section widening towards the plate
154b Section widening towards the ring element
154c Constriction
154d Side channel of the connecting device
155 Side channel of the bell cup
160 Engagement device, preferably for a tool

1.-48. (canceled)

49. Apparatus for disinfecting at least one room, in particular for one or more persons, preferably a dwelling room, storage room, cooling room or treatment room, for example a treatment room of a building, in particular a sickroom, a patient room and/or an operating room, with:
a rotatable bell cup for atomising a disinfectant,
a hollow drive shaft for driving the bell cup, and
a housing in which a drive unit for driving the drive shaft is accommodated and preferably the drive shaft is accommodated,
characterized in that
the housing has a plurality of cooling ribs, and/or
the drive shaft is provided with a labyrinth seal, preferably in order to prevent unwanted penetration of liquid into the housing.

50. Apparatus according to claim 49, characterized in that the cooling ribs project laterally outwards from the housing and/or are uniformly spaced from each other in the circumferential direction of the housing.

51. Apparatus according to claim 50, characterized in that the cooling ribs sectionally extend laterally outside along the drive shaft and/or parallel to the drive shaft.

52. Apparatus according to claim 51, characterized in that the cooling ribs sectionally extend laterally outside along the drive unit.

53. Apparatus according to claim 52, characterized in that the apparatus comprises at least one blower for generating a gas flow, preferably in order to transport and/or distribute disinfectant atomised by means of the bell cup in the room.

54. Apparatus according to claim 53, characterized in that gas guiding channels are disposed between the cooling ribs and the gas guiding channels serve for guiding of the gas flow.

55. Apparatus according to claim 54, characterized in that the labyrinth seal
is configured as a contactless labyrinth seal,
comprises a first section rotatable with the drive shaft and preferably a second static section and/or
performs its sealing function not until and/or only when the drive shaft rotates.

56. Apparatus according to claim 55, characterized in that
an intermediate gap is disposed between the first section and the second section, wherein an air barrier is formed in the intermediate gap by rotation of the first section; and/or
the first section and the second section do not touch each other.

57. Apparatus according to claim 56, characterized in that at least one elastic loading element is disposed between the second section and a bearing accommodated in the housing for rotatably mounting the drive shaft in order to act on the bearing.

58. Apparatus according to claim 57, characterized in that a hollow, preferably static inner pipe for supplying the disinfectant is disposed in the drive shaft.

59. Apparatus according to claim 58, characterized in that the inner pipe comprises a first portion and a second portion, wherein
the first portion has an outlet opening for the disinfectant, and/or
the second portion has an entrance opening for the disinfectant, runs between the drive unit and/or is carried in a mount for the housing.

60. Apparatus according to claim 59, characterized in that the second portion has at least one of the following features:
it has a greater external diameter than the first portion,
it has a greater wall thickness than the first portion,
it has a greater passage cross-section than the first portion.

61. Apparatus according to claim 60, characterized in that the housing is carried on a mount.

62. Apparatus according to claim 61, characterized in that
an O-ring seal is disposed between the mount and the housing, and/or
an O-ring seal is disposed between the mount and the inner pipe.

63. Apparatus according to claim 62, characterized in that a static part of the drive unit is provided with an O-ring seal and/or an inner face of a static part of the drive unit is protected by an inner tubular element made of corrosion-resistant plastic or by a corrosion-resistant coating.

64. Apparatus according to claim 63, characterized in that the apparatus has a ring structure that is openable and/or composed of several sections.

65. Apparatus according to claim 64, characterized in that the ring structure is openable and/or closable by means of at least one latch, plug and/or clamp connection.

66. Apparatus according to any of claim 65, characterized in that the ring structure has a concave and/or laterally outwards extending outer face, preferably in order to conduct further the gas flow laterally outwards.

67. Apparatus according to any of claim 66, characterized in that the ring structure comprises a rinsing agent channel for supplying rinsing agent and preferably for connecting to a rinsing agent line.

68. Apparatus according to claim 67, characterized in that the rinsing agent channel comprises an inlet hole and least one outlet hole, and
the inlet hole is disposed in the outer face and preferably comprises a connecting device for connecting to the rinsing agent line, and/or
the at least one outlet hole is disposed in an inner face of the ring structure and/or is directed towards the bell cup.
